(19) United States
(12) Patent Application Publication
(10) Pub. No.: US 2004/0016429 A1
(43) Pub. Date: Jan. 29, 2004

(54) Devices for dispensing powder products, such as medicines

(76) Inventor: Henri Bocquee, Paris (FR)

Correspondence Address: DOWELL & DOWELL PC, SUITE 309, 1215 JEFFERSON DAVIS HIGHWAY, ARLINGTON, VA, 22202

(21) Appl. No.: 10/312,866

(22) PCT Filed: Jul. 12, 2001
(86) PCT No.: PCT/FR01/02281

(30) Foreign Application Priority Data
Jul. 13, 2000 (FR) 00/09263

(51) Int. Cl. A61M 15/00

(52) U.S. Cl. 128/203.12

(57) ABSTRACT

The invention concerns a device for dispensing an active principle in powder form, comprising a box (2-104, 4-404); an adapter (6) for inhaling or blowing the active principle; a reservoir (10) for storing the active principle in powder form; a metering chamber (16, 116) enabling to draw a specific dose of the active principle, and integral with a part (12, 200) mobile between a drawing position wherein it communicates with the storage reservoir (10), and a position ready for use; means for driving (22, 24; 124) the metering chamber between its various positions; confinement means (32, 32a, 32b; 102b, 102c) for closing at least part of the metering chamber, in its ready position. In a position releasing the drawn dose of active principle, the metering chamber (16) is arranged to communicate, right through, on one side with an air supply chamber (34, 134), and on the other side with an inhalation opening (6), and the confinement means (32, 102) are mounted sliding relative to the box (1, 102; 4, 104) and arranged to close the passage passing through said metering chamber, in ready-for-use position, and to open said passage into the releasing position of said metering chamber.

The present relates to the domain of devices or apparatus for dispensing powdery active principles, of the medicine type, by pulmonary, buccal or nasal route.

According to the present invention, the terms used hereinafter in the description and claims have the following meaning:

active principle is understood to mean any product, in solid and divided form, particularly medicine or vaccination, capable of being administered to a patient with a view to curative or preventive treatment, or of diagnosis;

dose is understood to mean a predetermined quantity of active principle capable of being separated or drawn from a reservoir, during an elementary use of the device or apparatus according to the invention, with a view to a treatment.

Such devices are therefore adapted for the administration by pulmonary, buccal or nasal inhalation or insufflation. The administration of active principles in powder form is increasingly widespread and finds wider and wider domains of application.

It is envisaged to dispense to a patient an active principle in the form of powder for the treatment of certain diseases such as asthma or diabetes. For such applications, devices or apparatus should be used which make it possible, on the one hand, to ensure a precise dosage of the active principle and, on the other hand, to ensure a correct routing of the whole dose of active principle towards the zone of exchange and of absorption constituted by the lungs, the mucous membrane of the patient's mouth or nose.

Inhalation devices have already been developed.

It is known to make a device for administering active principle in the form of powder, comprising:

a box constituted by a first part and a second part, capable of moving with respect to each other in a relative movement;

an adapter for inhaling or blowing the active principle mounted on the box;

a reservoir for storing the active principle, disposed in the box;

a metering chamber for drawing a determined dose of active principle, and belonging to a part mobile or sliding between a drawing position in which it communicates with the storage reservoir, and a position ready for use;

means for driving the metering chamber between its different positions, under the effect of the relative movement of the two parts of the box;

and confinement means for closing at least in part the metering chamber filled with the drawn dose of the active principle, and in its standby position, said confinement means being mounted in the box.

Document WO 97/37707 for example discloses a powder inhaler comprising an air supply chamber, a conduit communicating with the chamber via an orifice and opening out in an inhalation mouthpiece, means for disposing a dose of powder in the conduit, and a detector of an aspiration created in the mouthpiece.

Such a device presents the drawback that it is very complex to make and consequently very expensive. Another drawback of such a device resides in the use of cells of which the filling by the active principle is ensured by gravity. Such a filling may be affected by a poor orientation or manipulation of the device.

The quantity of active principle drawn each time the device is used may no longer be constant. In addition, the dose of active principle drawn is located in a conduit communicating, on the one hand, with the supply chamber and, on the other hand, with an inhalation mouthpiece. The active principle drawn and intended for administration is therefore in contact with the air and ambient humidity. Such a contact risks altering the powdery consistency of the active principle and consequently its routing towards the lungs.

An administration device is also known, described in document U.S. Pat. No. 5,857,457, which comprises a box constituted by a first part and a second part capable of moving with respect to each other, in a relative movement in translation.

The device described likewise comprises an adapter for inhalation or blowing of the active principle, a reservoir for storing the active principle, a metering chamber making it possible to draw a determined dose of active principle and mobile between a position of drawing in which it communicates with the storage reservoir and a position of release of the drawn dose of the active principle, and means for driving the metering chamber under the effect of the relative movement of the two parts of the box. Such a device does not guarantee the maintenance of a precise dose of active principle in the metering chamber, awaiting its release.

Document U.S. Pat. No. 5,435,301 describes a device for administering active principle in the form of powder, by the pulmonary, buccal or nasal route, comprising a box constituted by a first part and a second part capable of moving with respect to each other in a relative movement, an adapter for inhalation or blowing of the active principle, a reservoir for storing the active principle, a metering chamber making it possible to draw a determined dose of active principle, and mobile between a position of drawing in which it communicates with the storage reservoir, and a position ready for use, and means for driving the metering chamber under the effect of the relative movement of the two parts of the box.

The metering chamber is integrated in the second part, i.e. the mobile part of the box. The device described comprises , furthermore, confinement means (in the form of flexible blade) making it possible to obturate the metering chamber containing the drawn dose, when said metering chamber is in stand-by position. The confinement means described are mounted in the box, on the one hand, and retractable with respect to the standby position, on the other hand, with the result that a bending of the confinement means makes it possible to release the dose contained in the metering chamber.

Such a device presents the drawback that the bending of the blade used as confinement means is provoked by the aspiration of the patient. Such an aspiration may be problematic for patients suffering from respiratory disorders or from asthma.

The present invention has for its object a device adapted for the administration of powdery active principle, making it possible to release with precision, in all circumstances, a dose of active principle already drawn from a reservoir.

In accordance with the present invention, in general, in a position of release of the drawn dose of the active principle, the metering chamber is arranged to communicate, right through, on one side with an air supply chamber, and on the other side with an inhalation or blowing mouthpiece. In correspondence, the confinement means, making it possible to obturate at least in part the metering chamber filled with the drawn dose of the active principle, in the stand-by position of said chamber, are mounted slidingly in the box, and arranged so as to close the through passage in the metering chamber, in ready-for-use position of said chamber, and to open this same passage into the releasing position of the same metering chamber.

Two principal and distinct forms of embodiment of the present invention are considered.

According to a first form of embodiment, the positions of the metering chamber, respectively of ready-for-use and of release of the drawn dose of active principle, are identical. In such a case, the confinement means are in that case independent of the mobile or sliding part comprising the metering chamber, and are retractable and arranged so as to close the passage through said metering chamber, in a so-called stand-by position of the aforementioned confinement means, and to open this same passage, in a position of release of the same confinement means.

According to a second form of embodiment, on the one hand, the positions of the metering chamber, respectively of ready-for-use and of release of the drawn dose, are distinct, and, on the other hand, in such a case, the confinement means comprise the mobile and sliding part incorporating the metering chamber, and a part of the box, in relation or cooperating with the sliding mobile part, completes the confinement means of the metering chamber, in the position ready-for-use of said chamber.

According to the first form of embodiment, for example, the confinement means are provided with a press button, accessible outside the box, allowing manual actuation thereof towards the position of release of the metering chamber.

According to the second form of embodiment, for example, the mobile or sliding part comprising the metering chamber, is provided with a push button accessible from outside the box, allowing, as

in the preceding case, manual actuation thereof towards the position of release of the metering chamber.

By way of example, the mobile part in which the metering chamber is made, slides in the box in a direction perpendicular to the direction of relative movement of the two parts of the box.

According to the afore-mentioned second form of embodiment, a grid optionally obturates the metering chamber in part, on the side of the mouthpiece for inhalation or blowing of the active principle.

For example, the means for driving the mobile or sliding part, comprising the metering chamber, comprise a drive arm mounted in the first part of the box, and a groove fast with the second part of the box, in which the drive arm is engaged.

The first part of the box may comprise a chamber for supply of air, on the one hand capable of being pressurized, compression means being in that case integrated in the box, and, on the other hand, capable of communicating with the metering chamber, on its filling side, when the mobile part is brought into the position of inhalation. In such a case, the mobile part comprises a cam whose end abuts on an inclined plane inside the first part of the box, to control its pivoting and a thrust on a valve controlling the air supply chamber, when this mobile part is brought into the position of inhalation; for example, the cam is mounted to pivot in the mobile part, and presents a part extending in the metering chamber.

Being question of the storage reservoir, the latter may be fast with the first part of the box, and present, on the one hand, a first open end, opening out on the mobile part, and if necessary on the metering chamber, and, on the other hand, a second end in which is introduced a piston pushing the active principle towards the first end, under the effect of the return force of an elastic means. This latter abuts for example on the second part of the box so as to increase the compression of said elastic means, when the two parts of the box move towards each other upon the relative movement thereof, in correspondence with the displacement and the filling of the

metering chamber. The end of the piston in contact with the active principle may present a protuberance, projecting into the metering chamber when the storage reservoir is empty, so as to block any displacement of the mobile or sliding part, comprising the metering chamber.

The administration device according to the invention presents the advantage of being able to fill the metering chamber with precision, independently of its orientation or its use in a place subject to shakings or vibrations, and in particular in a vehicle.

One advantage resides in the fact that the drawn dose remains confined in the metering chamber until the active principle is administered, directly by inspiration or under the effect of a charge of compressed air.

An important advantage is also obtained by the simple and reliable design of the device. In effect, the parts constituting the device are simple and easily interchangeable. It is also possible to modify the dosage of the active principle (quantity drawn upon each use) by modifying the volume of the metering chamber, and this by replacing the sliding part delimiting said metering chamber. Its filling other than by gravity makes it possible to guarantee a precise dosage of the active principle, and this whatever the volume of said metering chamber.

Another advantage of the device according to the invention resides in the delivery of a single dose of active principle independently of the number of rearmings or activations (i.e. of displacements of the metering chamber) made by the user. The single metering chamber, once filled, cannot in fact draw more active principle. The device according to the invention therefore makes it possible to guarantee a precise dosage.

A complementary advantage of the device according to the invention resides in the fact that the filling of the storage reservoir is effected only at the very end of the process of manufacture, i.e. when the device is entirely assembled. This assembly does not necessitate the same conditions of asepsis as the filling with the active principle, and may therefore be effected in an less expensive industrial environment, consequently reducing the manufacturing costs.

Other characteristics and advantages will also appear from the detailed description of non-limiting examples, given hereinafter with reference to the accompanying drawings, in which:

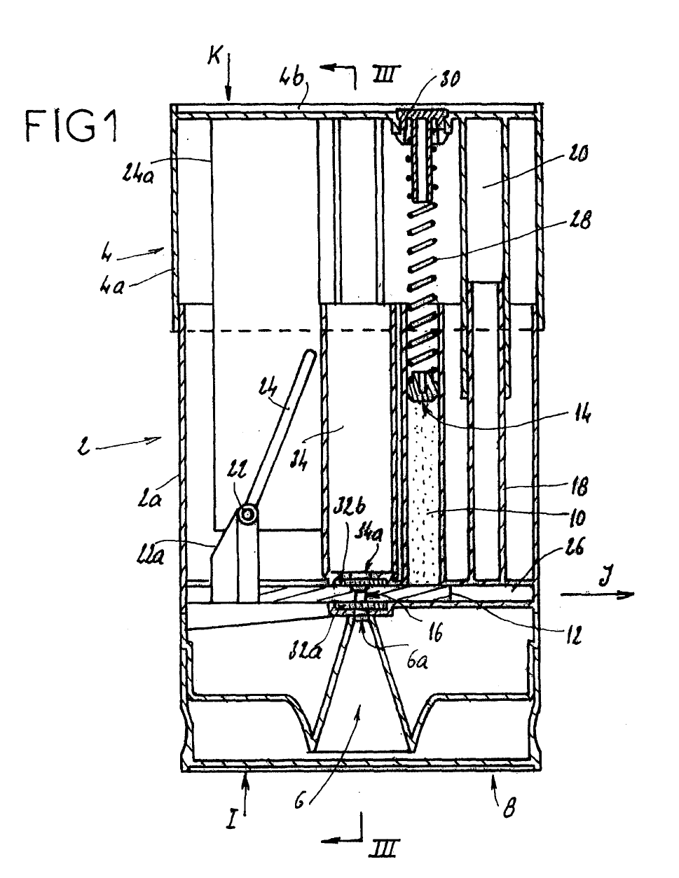

FIG. 1 shows a view in axial and vertical section of an administration device in accordance with a first form of embodiment of the invention.

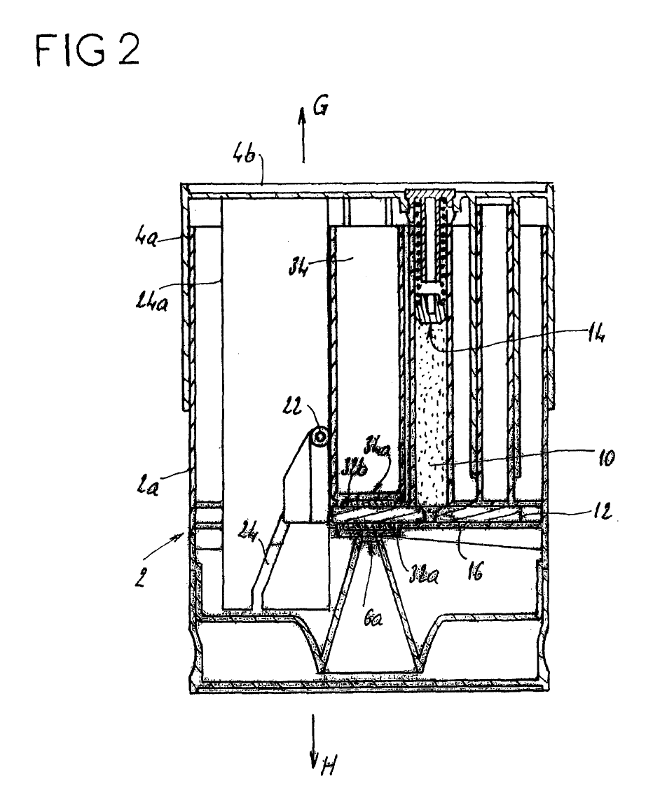

FIG. 2 shows the device of FIG. 1, in its position of charging.

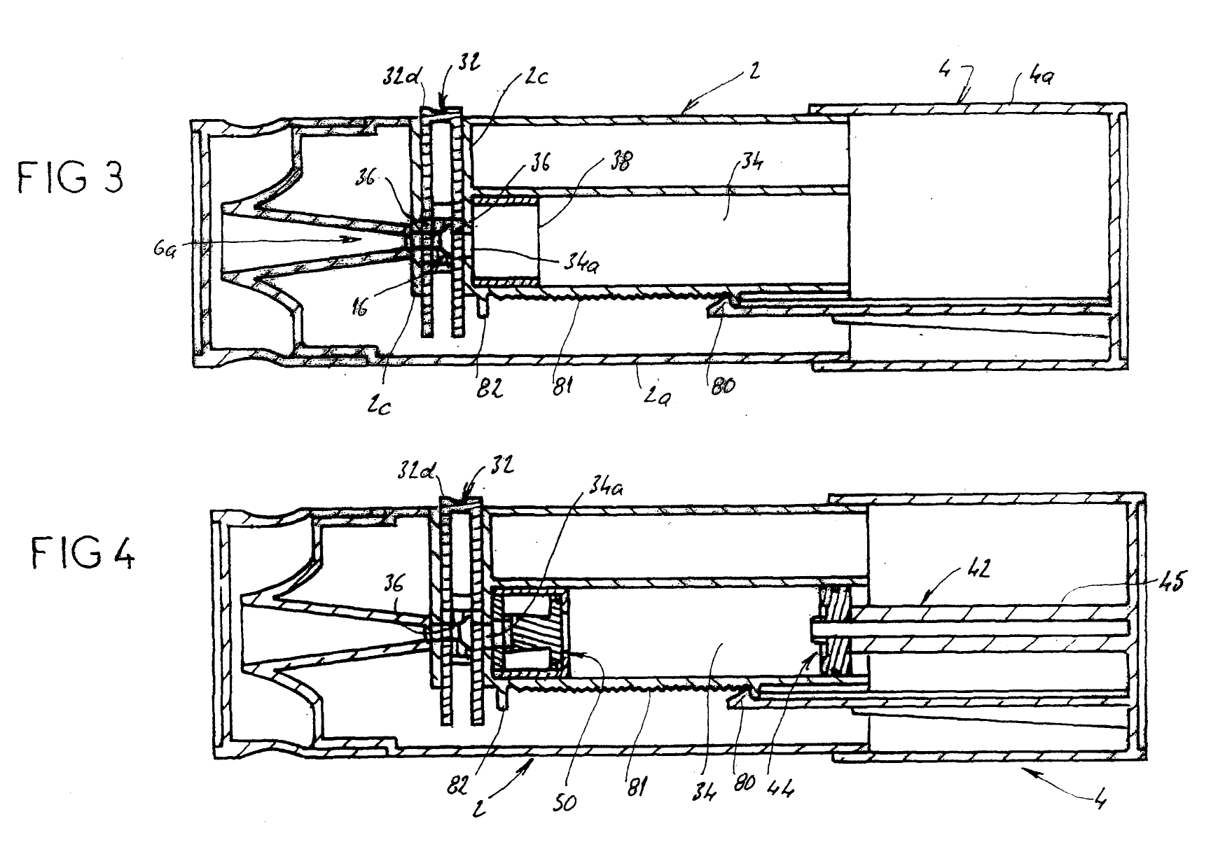

FIG. 3 shows a view in section along line III-III of the device of FIG. 1.

FIGS. 4 to 6 show a variant of the first form of embodiment of the invention.

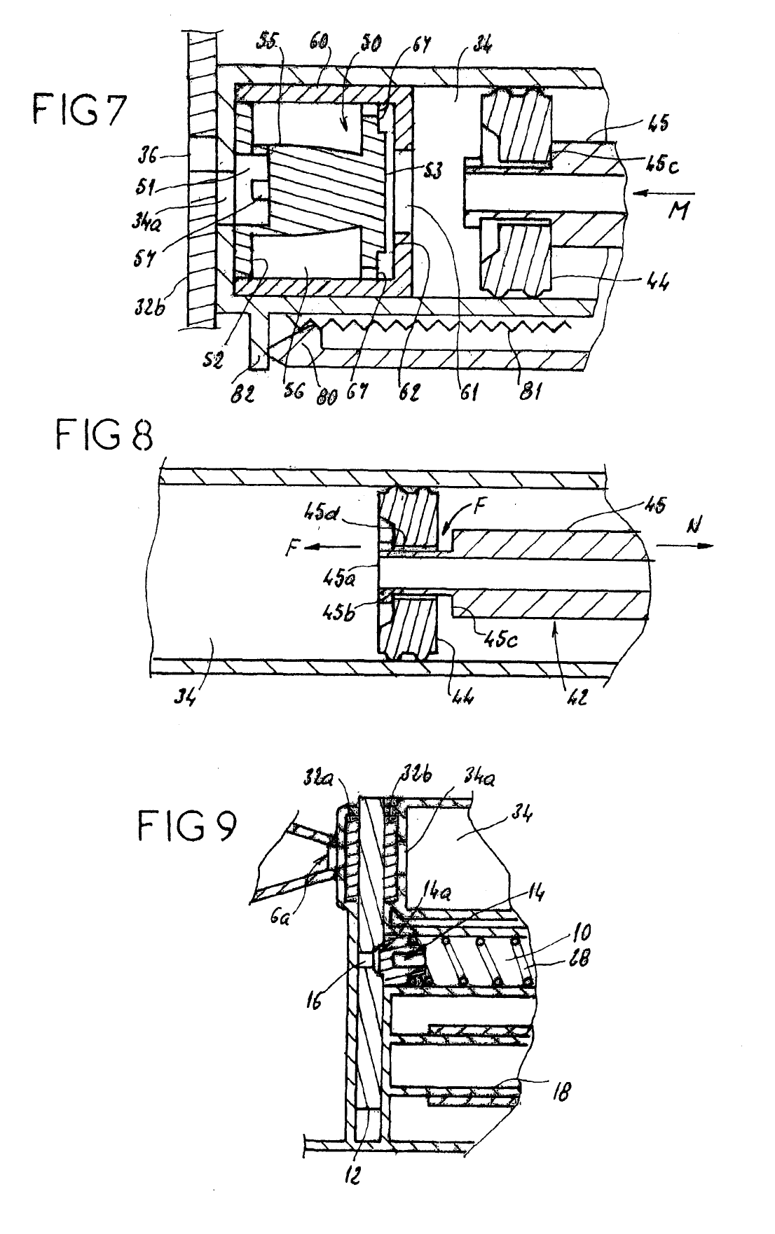

FIGS. 7 and 8 show enlarged details of FIGS. 4 to 6.

FIG. 9 shows another enlarged detail of the device shown in FIG. 1.

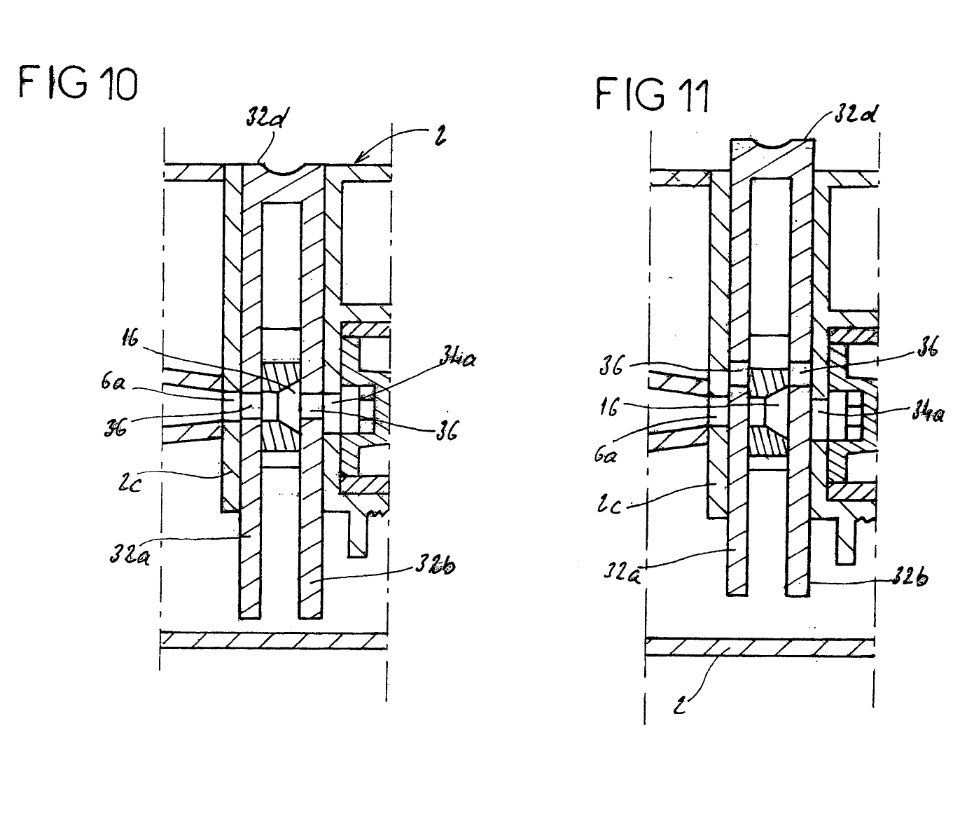

FIGS. 10 and 11 show other enlarged details of FIGS. 4 to 6.

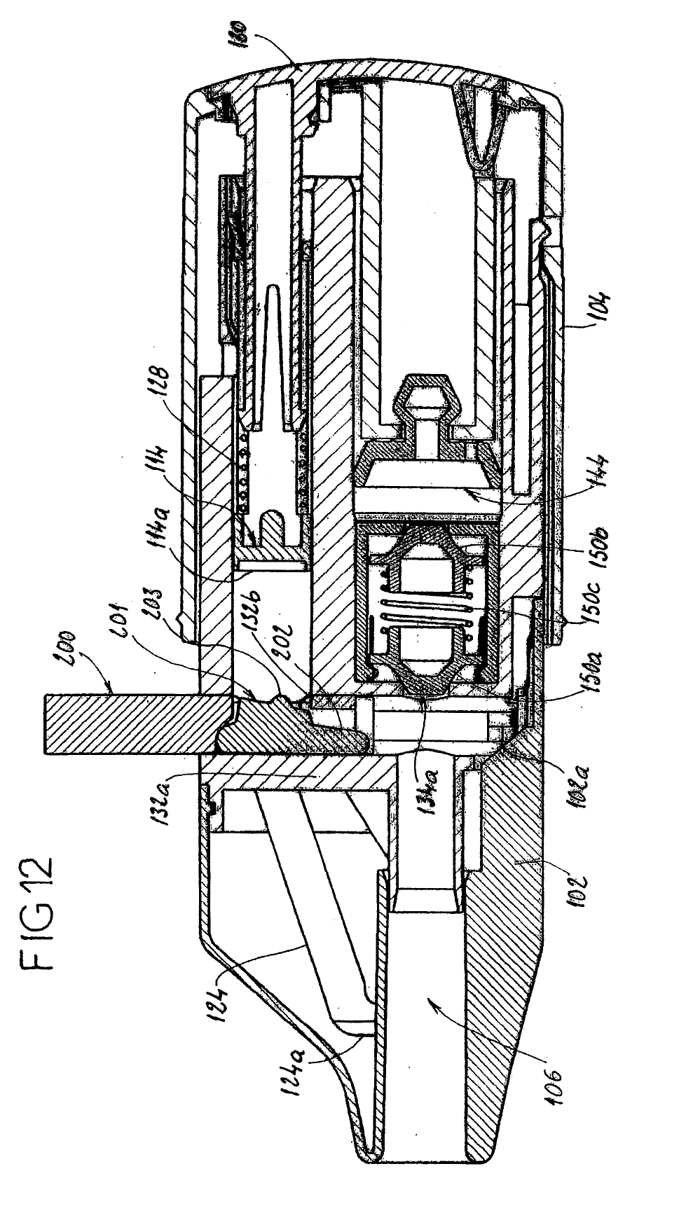

FIG. 12 shows the administration device in the second form of embodiment in accordance with the invention, seen in section.

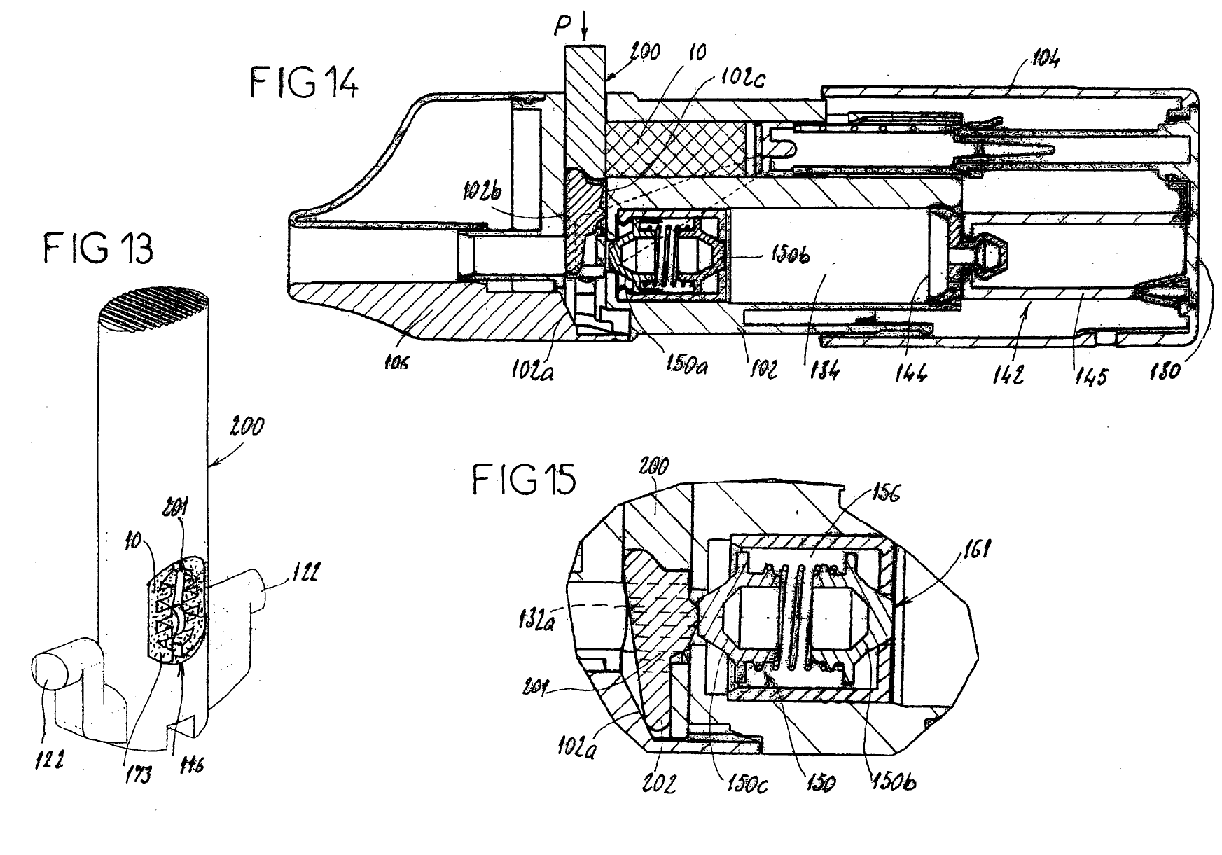

FIG. 13 shows a detail of FIG. 12.

FIG. 14 shows a partial view in section of the device of FIG. 12.

FIG. 15 shows another partial view in section of the device of FIG. 12.

The administration device shown in FIG. 1 is constituted by a box in two parts, namely a first box part 2 and a second box part 4, which are fitted in each other and capable of moving with respect to each other, in a relative movement of translation, parallel to the general axis of the device.

The first box part 2 presents, for example in transverse section, a circular or ellipsoidal form, defined by a peripheral wall 2a. The first box part 2 also comprises an administration adapter constituted in the present example by an inhalation mouthpiece 6. This latter is covered by a retractable lid 8. The first box part 2 also defines, inside, a storage reservoir 10 of the active principle. The storage reservoir 10 is for example of tubular shape, in the manner of a well. The storage reservoir 10 is obturated at one of its ends with the aid of a sliding part 12 or slide block, which is mounted mobile in translation in the first box part 2, at right angles to the axis of the device. The other end of the storage reservoir 10 is obturated with a piston 14, permanently returned towards the sliding part 12 by a spring 28.

The administration device according to the invention also comprises a metering chamber 16 making it possible to draw a determined dose of active principle with a view to administration thereof. This metering chamber 16, defined and included in the mobile or sliding part 12, is mobile between a position of drawing in which it communicates with the storage reservoir 10, and a stand-by position. FIG. 1 shows the metering chamber 16 in its stand-by position.

The second box part 4 presents, similarly to the first box part 2, in transverse section, a circular or ellipsoidal shape. The peripheral wall 4a of the second box part 4 covers at least in part a peripheral wall 2a of the first box part 2.

The peripheral wall 4a therefore slides over a portion of the peripheral wall 2a. The dimensions and the shape of the two corresponding and respective box parts 2, 4 have therefore been chosen accordingly.

The guiding of the relative slide between the first box part 2 and the second box part 4 is also ensured inside the box via a guide rod 18 fast with the first box part 2 and a guide tube 20 fast with the second box part 4, the rod 18 penetrating in adjusted manner in the tube 20.

Upon the relative displacement of said first and second box parts 2, 4, the guide rod 18 moves by sliding inside the guide tube 20. Means for limiting the slide (not shown) may also be provided between the first and the second box part 2, 4.

The sliding part 12 is mobile in the box by sliding and in a direction at right angles to the direction of the relative movement of the two box parts 2, 4. Means for driving the sliding part 12 and consequently the metering chamber, under the effect of the relative movement of the two box parts 2, 4, are also provided. These drive means comprise a drive arm 22, projecting transversely and fast with the sliding part 12, which is engaged in a groove 24 fast with the second part of the box 4. This groove 24 is for example a slit made in a longitudinal part 24a, fast with the second box part 4. The drive arm 22 comprises for example a pin sliding in said groove 24, via a roller, as the two box parts 2, 4 move relatively. The drive arm 22 is connected via a connecting part 22a to the sliding part 12.

A displacement of the second part of the box 4 in direction K and of the first part 2 in direction 1, thus makes it possible, via the drive means described hereinbefore, to drive the sliding part 12 in direction J, likewise schematized in FIG. 1. The sliding part 12 is guided in translation via a slideway 26 made in the first part of the box 2. In this way, a displacement of the metering chamber 16 is thus obtained, in a direction perpendicular to the direction of relative movement of the two box parts 2, 4, likewise in direction J.

The storage reservoir 10 fast with the first part of the box 2 therefore presents, on the one hand, a first open end opening out on the sliding part 12 and, if necessary, on the metering chamber 16, and, on the other hand, a second end in which is introduced the piston 14 pushing the stored powdery active principle towards the first end, under the effect of the return force of the spring 28 or an elastic means. This latter abuts on the second part of the box 4, so as to increase its compression when the two box parts 2, 4 move towards each other, during the relative movement thereof, and this simultaneously to the displacement of the metering chamber 16 towards the first end of the storage reservoir 10, with a view to the phase of filling of said metering chamber 16. The sliding part 12 thus makes it possible to obturate the storage reservoir 10 on the one hand, and to displace the metering chamber 16 between a position of filling or of drawing, and a standby position (this latter is shown in FIG. 1), on the other hand.

The spring 28, mounted in compression, on the one hand, on the piston 14 and, on the other hand, on a stopper 30 fixed intimately to the end face 4b of the second box part 4. This stopper 30 is preferably unable to be dismounted after its fixation and the filling of the storage reservoir 10. Such a filling is effected in the laboratory, under required conditions of asepsis. The user therefore has no access to the storage reservoir 10, without tampering with the device, this increasing safety of use thereof.

The spring 28 presents a length adapted to its introduction in the storage reservoir 10, and the piston 14 presents a shape and dimensions allowing it to ensure, on the one hand, its slide inside the storage reservoir 10 and, on the other hand, the required tightness with respect to the storage reservoir 10.

The administration device according to the invention also comprises confinement means 32 making it possible to obturate the metering chamber 16 containing the drawn dose of active principle, and this when said metering chamber 16 is in its stand-by position shown in FIG. 1. In its stand-by position, the metering chamber 16 is positioned between a registered expulsion opening 6a of the administration adapter 6, and a registered air-communication opening 34a, opening out in an air supply chamber 34, defined in the first box part 2. These two openings in that case define, in this stand-by position, a passage passing right through the metering chamber 16.

The confinement means 32 therefore make it possible to obturate the expulsion opening 6a and the air-communication opening 34a, simultaneously, and therefore to close the passage passing through the metering chamber 16, in order to isolate and confine the dose of active principle contained in the latter in its stand-by position.

The confinement means 32 are mounted mobile in translation in the box, on the one hand, and retractable with respect to the stand-by position, on the other hand. The confinement means 32 are mounted in the box, so that their direction of displacement is perpendicular to the direction of displacement of the metering chamber 16, and this in a plane at right angles to the direction of the relative movements of the two box parts 2, 4.

The displacement of the confinement means 32 thus makes it possible to release the dose of active principle contained in the metering chamber 16. This release is effected via an air flow obtained by any means, including by the simple aspiration by the patient, and passing respectively through the opening 34a, the metering chamber 16, and the expulsion orifice 6a, thus driving the active principle outside the box, which is then absorbed by the patient.

The airflow is either generated by a source of compressed air, or directly by the aspiration by the patient by buccal or nasal route.

FIG. 9 shows an enlarged detail of the administration device of FIG. 1, in which the storage reservoir 10 is empty. The end of the piston 14 usually in contact with the active principle presents a protuberance 14a projecting in the metering chamber 16 when the storage reservoir 10 is empty. The spring 28 makes it possible to push the piston 14 to the end of the storage reservoir 10, so that the protuberence 14a penetrates at least in part in the metering chamber 16 and blocks any displacement of the sliding part 12. It is thus impossible to use the administration device according to the invention when the storage reservoir 10 is empty, this constituting a safety of use for the patient in the administration of an active principle.

In accordance with an example of embodiment of the administration device according to the invention, the confinement means 32 are for example made in a hollow part or slide presenting two walls 32a, 32b positioned on either side of the sliding part 12 in order to obturate the metering chamber 16 when the latter is in its stand-by position. The walls 32a, 32b are traversed by release orifices 36, coaxial and aligned with the axis of the air supply chamber 34, in the relative position of the confinement means 32 and the sliding part 12, shown in FIG. 4. The release orifices 36, shown in particular in FIGS. 7, 10 and 11, are placed in

communication with the metering chamber 16 thanks to the displacement of said confinement means 32.

A specific slideway 2c is provided to that end so as to cause said confinement means 32 to slide, and to place the release orifices 36 in communication respectively with the expulsion orifice 6a and the air-communication opening 34a.

In accordance with an embodiment of the administration device according to the invention, the confinement means 32 are associated with an elastic return means (not shown), under the control of an elastic push button accessible from outside the first part of the box 2.

FIG. 2 shows the administration device according to the invention with the first and the second part of the box 2, 4, in an extreme position corresponding to a position of charging or of drawing of a dose of active principle. In this position, the metering chamber 16 is in communication with the storage reservoir 10, and the spring 28 is compressed so as to push the piston 14, thus contributing to correct filling of the metering chamber 16, and this whatever the orientation of the administration device.

FIG. 2 also shows the drive arm 22 in a first extreme position in the groove 24, and the walls 32a, 32b constituting the confinement means 32 which are in their position of isolation of the sliding part 12 with respect to the opening 34a for communication with the air and with respect to the expulsion orifice 6a. The charging or drawing of the dose of active principle is effected virtually instantaneously, and the user of the administration device according to the invention may then impart a relative movement of spacing-apart between the first and second box part 2, 4, by manually releasing the compression between the two parts 2 and 4, which move away from each other under the effect of the return of the elastic spring 28. This relative spacing-apart movement is represented by arrows G and H. The spacing apart movement terminated, the administration device is in its configuration of FIG. 1 again, in which the drive arm 22 is in a second extreme position in the groove 24, and the metering chamber 16 is isolated and confined between the walls 32a, 32b of the confinement means 32. The chamber 16 containing

the dose of active principle drawn from the storage reservoir 10 is then in a stand-by position, with a view to a use.

FIG. 3 shows an example of embodiment of the administration device of FIG. 1 in a section along line III-III. The air supply chamber 34 comprises for example a filter 38, making it possible, in the case of the airflow transporting the active principle being generated by the direct aspiration of the patient, to filter the ambient air aspired. This filter 38 is mounted by any means inside the air supply chamber 34. The metering chamber 16 is also in a stand-by position, i.e. isolated from the opening 34a for communication with the air, and from the expulsion orifice 6a, the release orifices 36 being offset with respect to the metering chamber 16.

FIG. 3 also shows the confinement means 32 with a push button 32d accessible from outside the first box part 2.

In another variant of the device according to the first form of embodiment of the invention (not shown in the Figures), the confinement means 32 are bereft of release orifices but present walls 32a, 32b of which the specific shape allows them to obturate the metering chamber 16 and to place said chamber 16 in communication with the expulsion orifice 6a as well as with the opening 34a.

FIGS. 4 to 6 show another example of embodiment of the adminstration device according to the first form of embodiment of the invention.

In this example of embodiment, the first box part 2 comprises the air supply chamber 34, capable of being pressurized. Air compression means 42 are integrated in the box parts 2, 4, and capable of communicating with the metering chamber 16, via one of the release orifices 36, when the latter communicates with the metering chamber 16.

An air compression chamber is thus produced with the air supply chamber 34, provided at one of its ends with the air compression means 42, and at the other of its ends with a valve 50 disposed in the vicinity of the opening 34a.

The air compression means 42 comprise a complementary piston 44 capable of moving in the

air supply chamber 34. The piston 44 is mounted on a rod 45 fast with the second part of the box 4, thus making it possible to displace the complementary piston 44 simultaneously to the displacement of the second part of the box 4.

The air compression means 42 are shown in greater detail in FIG. 8, which shows in enlarged manner the assembly of the piston 44 on the rod 45. This latter is for example constituted by a hollow rod presenting an annular shoulder 45c at its end engaged in the air supply chamber 34. The end 45a of the cylindrical rod 45 thus presents a reduced diameter on which is mounted the piston 44. The latter is engaged on said cylindrical end 45a of the rod 45. The inner diameter of the piston 44 is slightly larger than the outer diameter of the cylindrical end 45a so as to allow said piston 44 to slide on said cylindrical end 45a. This slide is effected between the annular shoulder 45c and a stop 45b of the rod 45. The shoulder 45c and the stop 45b make it possible to drive the piston 44 respectively in one of the opposite directions of displacement of the rod 45.

In this way, when the rod 45 moves so as to compress the air in the air supply chamber 34 (arrow M), the seal 45 is applied against the shoulder 45c so as to prevent any escape of air, via the clearance existing between said piston 44 and the cylindrical end 45a. Of course, the annular shoulder 45c presents a sufficient dimension to ensure this tightness.

On the other hand, when the rod 45 moves in the opposite direction, i.e. the one represented by arrow N in FIG. 8, it is the stop 45b provided at the end of the cylindrical end 45a which drives the piston 44. However, the stop 45b presents a configuration of the collar or rib type, or any other means making it possible, while being applied against the piston 44, to form an air inlet via the clearance existing between said piston 44 and the cylindrical end 45a. The circulation of air from outside to inside the air supply chamber 34 is materialized by arrows “F” in FIG. 8. The clearance existing between the piston 44 and the cylindrical end 45a is referenced by reference 45d.

FIG. 7 also shows an enlargement of the administration device in accordance with the first form of embodiment of

invention. It also represents a view in section showing in particular the arrangement of the pressurization valve 50 in the air supply chamber 34.

The valve 50 presents a barrel shape 55 comprising a deformable wall 52, 53 at each of its ends. The first end wall 52 is applied against the end of the air supply chamber 34. The opening 34a thus communicates with a well 51 formed in the valve 50. This latter is mounted in an assembly bush 60 presenting an air inlet 61 defined by an annular edge 62 and against which the second end wall 53 of the valve 50 is capable of coming into abutment. The first and the second ends 52, 53 of the valve 50 constitute a first and a second deformable wall 52, 53 shown in FIG. 7.

The valve 50 is advantageously made of an elastically deformable material so as to allow the second wall 53 to detach from the annular edge 62. In this way, when the piston 44 compresses the air contained in the air supply chamber 34, the valve 50 is deformed under the effect of the pressure and allows the wall 53 to detach from the annular edge 62. Air passages 67 are provided so as also to compress the air localized around the barrel 55 so as to produce a compression chamber 56, within the air supply chamber 34. This latter communicates with the well 51 via an additional passage 57, made by any means in the second end 52 of the valve 50.

The compression chamber 56, therefore of revolution, extends around the barrel 55. In this way, when the balance in pressure is attained between the air supply chamber 34 and the compression chamber 56, the second branch 53 moves again towards the annular edge 62 to resume its initial shape not deformed elastically. A compression chamber 56 is in that case obtained, which is perfectly tight with respect to the outside medium, and the rod 45 may again move in the direction of arrow “N” (cf. FIG. 8) so as to allow air to penetrate again in the air supply chamber 34 as schematically shown in FIG. 8.

The valve 50 thus makes it possible to store compressed air in the compression chamber 56, up to the moment when the corresponding release orifice 36 is displaced in order to come into communication with the opening 34a and the metering chamber 16, as shown in FIG. 10.

The pressure obtained by the administration device according to the invention is sufficient to expel the dose of active principle contained in the metering chamber 16 and to route it towards the lungs, the buccal or nasal mucous membranes. The air inlet 61 may also be associated with a filter 38 (not shown).

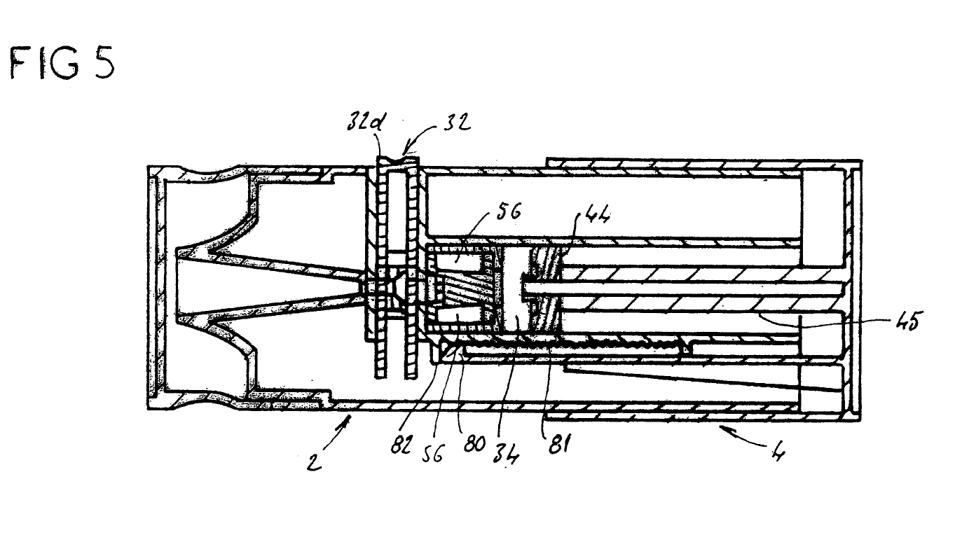

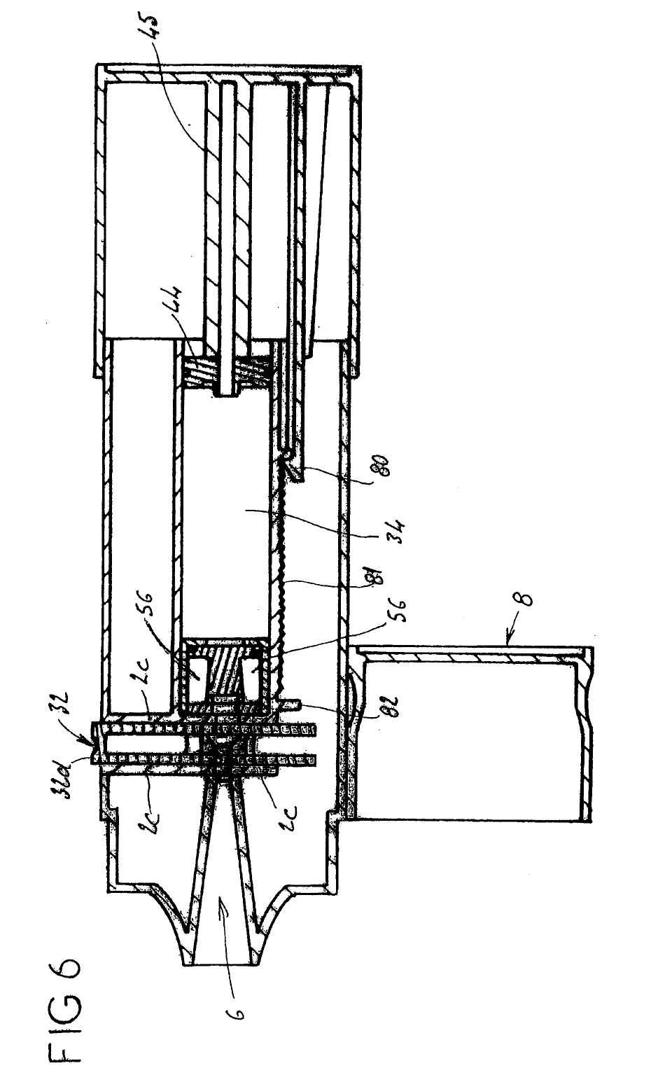

FIGS. 4 to 6 schematically show the different phases of use of the administration device in accordance with the first form of embodiment of the invention. Thus FIG. 5 schematically shows the pressurization of the compression chamber 56 by the displacement of the piston 44 and of the rod 45. FIG. 6 then shows the administration device ready to be used. In effect, the compression chamber 56 is pressurized, despite the return of piston 44 into its initial position. The air supply chamber 34 is then again at atmospheric pressure. It then suffices to exert a pressure or to displace the confinement means 32 in order to release the compressed air stored in the compression chamber 56.

According to an example of embodiment (not shown in the Figures), the pressurization valve 50 may also be constituted by a piston mounted against a return force of a spring, which is disposed between said piston and the end of the air supply chamber 34. The piston is then applied in rest position against the annular edge 62.

According to an example of embodiment shown in FIGS. 3 and 4, the first box part 2 comprises a rack 80 on which is engaged the end of a flexible arm 81 fast with the second box part 4 (cf. FIG. 4, for example). The engagement of the end of the flexible arm 81 on the rack 80 makes it possible, whenever the device is armed, i.e. whenever the metering chamber 16 is charged, to generate a sound signal.

The end of the flexible arm 81 is also capable of coming into abutment against a stop 82 fast with the first box part 2, thus constituting an end-of-stroke of the relative movements of the box parts 2, 4.

Other known devices which enable compressed air to be obtained may also be used in an administration device according to the invention.

In the following specification, and more precisely in accordance with the second form of embodiment of the invention shown in FIGS. 12 to 14, functionally identical parts and elements conserve their name but bear references increased by 100.

Functioning which is unchanged with respect to FIGS. 1 to 11 is no longer described. Reference may be made to the description of said FIGS. 1 to 11.

The structurally and/or functionally unchanged parts are no longer described and conserve their name with the same reference, unless duly mentioned.

The device according to the invention, shown in FIG. 12, comprises a mobile or sliding part 200, displaceable at right angles to the displacement of the two parts of the box 102, 104. The sliding part 200 comprises a cam 201, mounted to pivot in said sliding part 200. This latter also presents a through cavity defining the metering chamber 116, which is thus likewise mobile with the part 200, by sliding in the box 102, in a direction at right angles to the direction of relative movement of the two box parts 102, 104. The cam 201 extends in part in the metering chamber 116. The dose of active principle drawn thus fills the metering chamber 116 on either side of the cam 200.

FIG. 13 shows for reasons of clarity a partial filling of the metering chamber 116. A grid 173, partially obturating the metering chamber 116 towards the adapter 6 and the confinement walls 102b and 102c belonging to the first part 102 of the box; obturates the metering chamber 116 on its filling side, when said metering chamber 116 is in its stand-by position (cf. FIG. 14). The grid 173 is advantageously slit at its centre to allow the cam 201 to pivot. This latter is also displaced by a translation connected with the slide of the part 200.

In an extreme position of the sliding part 200, corresponding to the release of the drawn dose of the active principle, shown in FIG. 15, the end 202 of the cam 201 abuts on an inclined plane inside the first part of the box 102, in order to control its pivoting and a thrust on the valve 150. This extreme position corresponds to a position of inhalation and to a release of the compressed air stored in the compression chamber 156. The valve 150, whose functioning is similar to that of valve 50, is for example made of two flap valves 150a, 150b assembled together by a compression spring 150c returning them respectively in opposite direction; the flap valve 150a closes the compression chamber 156, by means of an appropriate seat, towards the mouthpiece 106, and the flap valve 150b towards the air supply chamber 134.

The sliding or mobile part 200 symmetrically presents two drive arms 122 engaged in corresponding inclined grooves 124 fast with the second box part 104. The drive arms 122 and the inclined grooves 124 thus form the means for driving the part 200. Upon the relative movement of the two box parts 102, 104, it is the inclined grooves 124 which make it possible to displace the sliding part 200, and consequently the metering chamber 116 between a position of drawing (cf. FIG. 12) and a stand-by position.(cf. FIG. 14).

In this stand-by position, the confinement of the drawn dose is ensured, and the confinement means 102b, 102c are rendered inoperative only with an action or pressure on the sliding part 200 in the direction of arrow “P”, by the user. Such an action generates a complementary displacement of the metering chamber 116 from its stand-by position towards the position of release of the drawn dose of the active principle, and therefore of inhalation (cf. FIG. 15), in which a projecting part 203 of the cam 201 abuts on the valve 150a, thus releasing the compressed air available in the chamber 156. The released airflow then drives the active principle contained in the metering chamber 116 through the grid 132a in the adapter 106.

The complementary displacement of the sliding or mobile part 200, from the stand-by position towards the position of inhalation is rendered possible by the shape of the inclined grooves 124. These latter thus comprise at one of their ends a portion 124a at right angles allowing a complementary slide of the drive arms 200a, under the effect of a manual thrust on the sliding part 200.

Functioning of the administration device shown in FIGS. 12 to 15 is specified hereinafter.

The user draws a dose of active principle by bringing the two box parts 102, 104 as close as possible by relative slide, as shown in FIG. 12. The metering chamber 116 (not shown) thus presents its filling opening in communication with the storage reservoir 10. A maximum spacing apart in return of the two box parts 102, 104 (cf. FIG. 14) places the metering chamber 116 in stand-by position, and thus ensures the confinement of the drawn dose. A manual thrust on the sliding part 200 in the direction of arrow “P” (cf. FIG. 14) then makes it possible, via the cam 201, to release the stored compressed air and to generate an airflow routing the active principle through the metering chamber 116.

When the user no longer exerts any pressure on the sliding part 200, this latter rises slightly in the direction opposite the thrust, thanks to the pressurization valve 105a, which exerts an elastic return force on the end 202 of the cam 201, in abutment on the inclined plane 102a.

This brings about a rise of the drive arms 122 in the right-angled guiding part 124a. After this rise, it is the inclined grooves 124, via a fresh relative movement of approach of the two box parts 102, 104, which make it possible to return the sliding part 200 and consequently the metering chamber 116, into its draw position again.

In summary, the two forms of embodiment of the invention have in common the characteristic whereby, in the position of release of the drawn dose of the active principle, the metering chamber 16 or 116 is arranged to communicate, in completely through manner, on one side with the air supply chamber 34 or 134, or more precisely the compression chamber 56 or 156, and on the opposite side with a mouthpiece 6 or 106 for inhalation or blowing. In correspondence, the confinement means 32 or 102b-c are mounted slidingly with respect to the box 2-4, or 102-104, or vice versa, and arranged so as to close the passage traversing the afore-mentioned metering chamber, in the position of ready-for-use of the mobile part 12 or 200, and to open this same passage in the position of release of the afore-mentioned metering chamber, more precisely of the mobile part 12 or 200.

According to the first form of embodiment, the positions of the metering chamber 16, respectively ready-for-use (cf. FIG. 1) and of release of the drawn dose (cf. FIG. 6), are identical. In that case, the confinement means 32 are independent of the mobile or sliding part 12, and are retractable and arranged so as to close the passage through the metering chamber 16, in a first so-called stand-by position (cf. FIG. 3) of the afore-mentioned confinement means, and to open the same through passage, in a second so-called release position (cf. FIG. 4) of the same confinement means.

According to the second form of embodiment of the invention, the positions of the metering chamber 116, ready-for-use (cf. FIG. 14) and of release (cf. FIG. 15) of the drawn dose, are respectively distinct. In that case, a part 102b-102c of the box 102, in relation with the sliding mobile part 200, completes the confinement means of the chamber 116, in the position of the latter ready-for-use.

“Confinement” is understood to mean an at least partial isolation of the metering chamber.

1. Device for adminstering an active principle in powder form, by pulmonary, buccal or nasal route, comprising:
a box constituted by a first part (2, 102) and a second part (4, 104), capable of moving with respect to each other in a relative movement;
an adapter (6) for inhaling or blowing the active principle mounted on the box;
a reservoir (10) for storing the active principle, disposed in the box;
a metering chamber (16, 116) for drawing a determined dose of active principle, and belonging to a part (12, 200) mobile between a drawing position in which it communicates with the storage reservoir (10), and a position ready for use;
means (22, 24; 124) for driving the metering chamber between its different positions,
and confinement means (32, 32a, 32b; 102b, 102c) for closing at least in part the metering chamber filled with the drawn dose of the active principle, in its standby position; characterized in that, in a position of release of the drawn dose of the active principle, the metering chamber (16, 166) is arranged to communicate, right through, on one side with an air supply chamber (34, 134), and on the other side with an inhalation or blowing mouthpiece (6, 106), and the confinement means (32, 102) are mounted slidingly with respect to the box (2, 102; 4, 104), and arranged so as to close the passage passing through said metering chamber, in ready-for-use position of said chamber, and to open this same passage into the releasing position of said metering chamber.

2. Device according to claim 1, in which the positions of the metering chamber (16), respectively ready-for-use and for release of the drawn dose, are identical, characterized in that the confinement means (32) are independent of the mobile part (12), and are retractable and arranged so as to close the passage through said metering chamber, in a stand-by position of said confinement means, and to open said passage, in a position of release of said confinement means.

3. Device according to claim 1, characterized in that, on the one hand, the positions of the metering chamber (116), ready-for-use and for release of the drawn dose, are respectively distinct, and, on the other hand, the confinement means comprise a mobile and sliding part (200), a part (102b, 102c) of the box (102), in relation with said mobile part (200) completing the confinement means of the metering chamber (116), in the position of the latter ready for use.

4. Device according to claim 1, characterized in that the confinement means (32) are provided with a push button, accessible outside the box, allowing their manual actuation towards the position of release of the metering chamber (16).

5. Device according to claim 3, characterized in that the mobile part (12) is provided with a push button, accessible outside the box, allowing its manual actuation towards the position of release of the metering chamber (116).

6. Device according to claim 1, characterized in that the mobile part (12, 200) slides in the box in a direction perpendicular to the direction of relative movement of the two box parts (2-4, 102-104).

7. Device according to claim 3, characterized in that a grid (173) partially obturates the metering chamber (116) on the adapter (106) side.

8. Device according to claim 1, characterized in that the drive means of the mobile part (12, 200) comprise a drive arm (22, 122) mounted in the first part (2) of the box, and a groove (24, 124) fast with the second part (4) of the box, in which said drive arm is engaged.

9. Administration device according to claim 1, characterized in that the first box part (2, 102) comprises an air supply chamber (34, 134), on the one hand, capable of being pressurized, air compression means (42, 142) being integrated in the box, and, on the other hand, capable of communicating with the metering chamber (16, 116), on its filling side, when the mobile part (12, 200) is brought into the position of inhalation.

10. Device according to claim 9, characterized in that the mobile part (200) comprises a cam (201) of which the end (202) abuts on an inclined plane (102a) inside the first box part (102) to control its pivoting and a thrust on a valve (150) controlling the air supply chamber (134), when said mobile part (200) is brought into the position of inhalation.

11. Device according to claim 10, characterized in that the cam (201) is mounted to pivot in the mobile part (200), and presents a part extending in the metering chamber (116).

12. Device according to claim 1, characterized in that the storage reservoir (10) is fast with the first box part (12, 102) and presents, on the one hand, a first open end, opening out on the mobile part (12, 200), and if necessary on the metering chamber (16, 116) and, on the other hand, a second end in which is introduced a piston (14, 114) pushing the active principle towards the first end, under the effect of the return force of an elastic means (28, 128).

13. Device according to claim 12, characterized in that the elastic means (28, 128) abuts on the second box part (4, 104), so as to increase the compression of said elastic means, when the two box parts (2-4, 102-104) move towards each other during the relative movement thereof, in register with the displacement and the filling of the metering chamber (16, 116).

14. Device according to claim 13, characterized in that the end of the piston (14, 114), in contact with the active principle presents a protuberance (14a, 114a), projecting in the metering chamber (16, 116) when the storage reservoir (10) is empty, so as to block any displacement of the mobile part (12, 200), and consequently of the metering chamber (16, 116).

15. Device according to claim 1, characterized in that the mouthpiece (6, 106) constitutes a buccal inhaler or blower.

16. Device according to claim 1, characterized in that it comprises a nasal adapter in order to constitute a nasal blower.
